(19) United States
(12) Patent Application Publication
(10) Pub. No.: US 2022/0125403 A1
(43) Pub. Date: Apr. 28, 2022

(54) ULTRASONIC TOMOGRAM GENERATION METHOD, ULTRASONIC TOMOGRAM GENERATION APPARATUS, AND PROGRAM

(71) Applicants: National University Corporation University of Toyama, Foyama-shi, Foyama (JP); GENERAL INCORPORATED ASSOCIATION MEDICAL INNOVATION CONSORTIUM, Tokyo (JP)

(72) Inventor: Hideyuki HASEGAWA, Toyama-shi (JP)

(73) Assignee: GENERAL INCORPORATED ASSOCIATION MEDICAL INNOVATION CONSORTIUM, Tokyo (JP)

(21) Appl. No.: 17/427,989

(22) PCT Filed: Feb. 2, 2020
(86) PCT No.: PCT/JP2020/003839

§ 371 Date: Aug. 3, 2021

(30) Foreign Application Priority Data
Feb. 4, 2019 (JP) 2019-017723

(51) Int. Cl. A61B 8/14; A61B 8/08; A61B 8/00; G01S 7/52

(52) U.S. Cl. CPC A61B 8/14; G01S 7/52077; A61B 8/4444; A61B 8/5207

(57) ABSTRACT

Provided is a technique for improving spatial resolution contrast of an ultrasonic tomogram compared with a method based on a coherence between echo signals. The present invention provides an ultrasonic tomogram generation method including: an estimation step SA100 of estimating noise in echo signals of M channels output from an ultrasonic probe, which receives echoes of ultrasonic waves emitted from M (being a natural number of 2 or more) ultrasonic transducers and outputs an echo signal, and calculating a weight coefficient for emphasizing an echo from a reception focus according to a signal-to-noise ratio in the echo signals of the M channels; and a generation step SA110 of generating a beamformer representing an ultrasonic tomogram from the echo signals of the M channels, using the weight coefficient calculated in the estimation step SA100.

### TECHNICAL FIELD

The present invention relates to a technique for generating an ultrasonic tomogram.

### BACKGROUND ART

Ultrasonic diagnosis is a method of non-invasively measuring a tomogram in a subject's body, and is widely used in a medical field. Spatial resolution and contrast of an ultrasonic tomogram obtained by ultrasonic diagnosis are important factors that directly relates to diagnosis accuracy. For this reason, various techniques have been proposed to improve the spatial resolution and contrast of the ultrasonic tomogram, and includes a technique disclosed in Non-Patent Literature 1 as an example. In the technique disclosed in Non-Patent Literature 1, the orientation resolution and contrast of the ultrasonic tomogram are improved based on coherence between echo signals received by an array-type ultrasonic transducer formed of a plurality of ultrasonic transducers.

### CITATION LIST

### Non-Patent Literature

Non-Patent Literature 1: Ultrasound imaging method based on coherence between received signals, P.-C. Li and M. -L. Li, “Adaptive imaging using the generalized coherence factor,” IEEE Trans. Ultrason. Ferroelectr. Freq. Control, vol. 50, no. 2, pp. 128-141, 2003.

Non-Patent Literature 2: H. Hasegawa and H. Kanai, “Effect of element directivity on adaptive beamforming applied to high-frame-rate ultrasound,” IEEE Trans. Ultrason. Ferroelectr. Freq. Control, vol. 62, no. 3, pp. 511-523, 2015.

### SUMMARY OF INVENTION

### Technical Problem

As described above, since the spatial resolution and contrast of the ultrasonic tomogram directly relates to diagnosis accuracy, higher spatial resolution and contrast thereof are more preferable.

The present invention has been made in view of the above circumstances, and aims to provide a technique for improving spatial resolution and contrast of an ultrasonic tomogram compared with a method using coherence between echo signals.

### Solution to Problem

In order to solve the above problem, the present invention is to provide an ultrasonic tomogram generation method including: an estimation step of estimating noise in echo signals of M channels output from an ultrasonic probe, which receives echoes of ultrasonic waves emitted from M (being a natural number of 2 or more) ultrasonic transducers and outputs an echo signal, and calculating a weight coefficient for emphasizing an echo from a reception focus according to a signal-to-noise ratio in the echo signals of the M channels; and a generation step of generating a beamformer representing an ultrasonic tomogram from the echo signals of the M channels, using the weight coefficient calculated in the estimation step.

Although details will be described below, according to the present invention, it is possible to improve the spatial resolution and contrast of the ultrasonic tomogram compared with the method using the coherence between the echo signals.

In the ultrasonic tomogram generation method, more preferably, in the estimation step: a cumulative element signal uₘ may be obtained by cumulation up to a m-th echo signal sₘ obtained by addition of a delay in delay-and-sum beamforming to the echo signals of the M channels; a modeling element signal may be modeled as Uₘ=m×y+n using a direct component “y” included in the echo signal sₘ and a bias “n” caused by additional noise; values of the y and n may be set such that a mean squared error α of the cumulative element signal and the modeling element signal is minimized; a minimum value of the mean squared error α may be calculated using the set values of the y and n; and the weight coefficient may be calculated from the minimum value and the set value of the y, and in the generation step, the set value of the y may be multiplied by the weight coefficient to generate the beamformer representing the ultrasonic tomogram.

In the ultrasonic tomogram generation method, more preferably, in the estimation step, the weight coefficient may be calculated from a root mean square of an integral value nₘ of noise contained up to a m-th echo signal sₘ obtained by addition of a delay in delay-and-sum beamforming to the echo signals of the M channels and a root mean square of an average Y_{DAS} of the echo signal sₘ after delay compensation, and in the generation step, the average Y_{DAS} may be multiplied by the weight coefficient to generate the beamformer representing the ultrasonic tomogram.

In order to solve the above problem, the present invention is to provide an ultrasonic tomogram generation apparatus including: estimation means for estimating a signal-to-noise ratio in echo signals of M channels output from an ultrasonic probe, which includes M (being a natural number of 2 or more) ultrasonic transducers, receives echoes of ultrasonic waves emitted from the respective ultrasonic transducers, and outputs an echo signal, and calculating a weight coefficient for emphasizing an echo from a reception focus according to a signal-to-noise ratio in the echo signals of the M channels; and generation means for generating a beamformer representing an ultrasonic tomogram from the echo signals of the M channels, using the weight coefficient calculated by the estimation means.

In order to solve the above problem, the present invention is to provide a program causing a computer to function as: estimation means for estimating a signal-to-noise ratio in echo signals of M channels output from an ultrasonic probe, which includes M (being a natural number of 2 or more) ultrasonic transducers, receives echoes of ultrasonic waves emitted from the respective ultrasonic transducers, and outputs an echo signal, and calculating a weight coefficient for emphasizing an echo from a reception focus according to a signal-to-noise ratio in the echo signals of the M channels; and generation means for generating a beamformer representing an ultrasonic tomogram from the echo signals of the M channels, using the weight coefficient calculated by the estimation means.

### BRIEF DESCRIPTION OF DRAWINGS

FIG. 1 is a block diagram showing a configuration example of an ultrasonic medical system 1 including an ultrasonic tomogram generation apparatus 20 according to an embodiment of the present invention.

FIG. 2 is a flowchart showing a flow of signal processing to be executed by a signal processing unit 230 of the ultrasonic tomogram generation apparatus 20.

FIG. 3 is a view showing an imaging result of a point target for evaluating spatial resolution of an ultrasonic tomogram.

FIG. 4 is a view showing an imaging result of a phantom for evaluating a contrast of the ultrasonic tomogram.

### DESCRIPTION OF EMBODIMENT

An embodiment of the present invention will be described below with reference to the drawings.

### A. EMBODIMENT

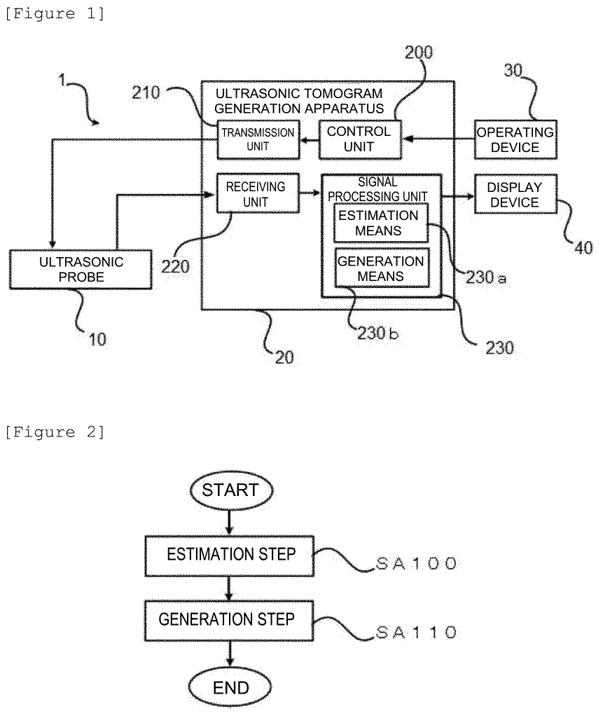

FIG. 1 is a diagram showing a configuration example of an ultrasonic medical system 1 including an ultrasonic tomogram generation apparatus 20 according to an embodiment of the present invention. The ultrasonic medical system 1 is a system configured to capture non-invasively an ultrasonic tomogram in a subject's body in a medical field. As shown in FIG. 1, the ultrasonic medical system 1 includes an ultrasonic probe 10, an operating device 30, and a display device 40 which are connected to the ultrasonic tomogram generation apparatus 20 via signal lines, respectively, in addition to the ultrasonic tomogram generation apparatus 20.

The ultrasonic probe 10 includes an array-type ultrasonic transducer formed of a plurality of ultrasonic transducers. In the ultrasonic medical system 1 of the present embodiment, a linear array probe (PU-0558: Ueda Japan Radio Co., Ltd.) is used as the ultrasonic probe 10 in which M (being a natural number of 2 or more) ultrasonic transducers are arranged at intervals of 0.1 mm. Each of the plurality of ultrasonic elements emits ultrasonic waves toward an inspection site of the subject under control of the ultrasonic tomogram generation apparatus 20, receives echoes of the ultrasonic waves, and outputs echo signals.

The ultrasonic tomogram generation apparatus 20 causes the ultrasonic probe 10 to transmit ultrasonic waves, and also performs signal processing on a signal output from the ultrasonic probe 10 to generate image data. The operating device 30 includes a pointing device such as a mouse and a keyboard. The operating device 30 is a device for causing a user (for example, an inspection technician who performs various operations for ultrasonic diagnosis) of the ultrasonic medical system 1 to perform various input operations on the ultrasonic tomogram generation apparatus 20. The display device 40 is, for example, a liquid crystal display. The display device 40 displays an image according to the image data output from the ultrasonic tomogram generation apparatus 20.

As shown in FIG. 1, the ultrasonic tomogram generation apparatus 20 includes a control unit 200, a transmission unit 210, a receiving unit 220, and a signal processing unit 230. Although not shown in detail in FIG. 1, the ultrasonic tomogram generation apparatus 20 also includes a storage unit (for example, a hard disk) that stores various software such as an OS (Operating System).

The control unit 200 is a CPU (Central Processing Unit), for example. The control unit 200 functions as a control center of the ultrasonic tomogram generation apparatus 20 by executing the software stored in the storage unit, and controls the operation of each unit. More specifically, the control unit 200 controls the operation of each unit such that an ultrasonic tomogram is generated by an acquisition sequence for each line as in the conventional way.

The ultrasonic probe 10 is connected to the transmission unit 210 and the receiving unit 220 via signal lines. The transmission unit 210 performs D/A conversion on transmission data sent from the control unit 200 to generate a transmission signal, and sends the transmission signal to the M ultrasonic transducers included in the ultrasonic probe 10. Thereby, each of the M ultrasonic transducers included in the ultrasonic probe 10 emits ultrasonic waves. The receiving unit 220 performs A/D conversion on the echo signal output from each of the plurality of ultrasonic transducers included in the ultrasonic probe 10, further gives a delay to the echo signal for the purpose of delay compensation, and sends the echo signal to the signal processing unit 230. In the present embodiment, the delay given to the echo signal by the receiving unit 220 is a delay based on delay-and-sum beamforming (hereinafter, DAS beamforming) which is a conventional ultrasonic tomogram generation method.

Assuming that the echo signal output from the m (m=0, 1, 2, . . . M−1)-th ultrasonic transducer of the ultrasonic probe 10 and delayed by the receiving unit 220 is defined as sₘ, the echo signal obtained by the M ultrasonic transducers included in a reception aperture of the ultrasonic probe 10 is represented by a vector S shown in Equation 1 to be described below. After the delay compensation, the echo from a reception focus included in the vector S becomes a direct current (DC) component which crosses over the reception aperture. Therefore, in the conventional DAS beamforming, a beamformer (that is, a beamformer representing an ultrasonic tomogram) Y_{DAS} corresponding to the echo from a reception focus y is represented, as an average of the echo signal sₘ after the delay compensation, by Equation 2 to be described below.

$$\begin{matrix}s = {[ {s}_{0} {s}_{1} … {s}_{M - 1} ]}^{T} & [ Equation 1 ] \\ {Y}_{DAS} = \frac{1}{M} {\sum }_{i = 0}^{M - 1} {s}_{i} & [ Equation 2 ]\end{matrix}$$

On the other hand, the signal processing unit 230 performs signal processing (beamforming processing based on the signal-to-noise ratio), which remarkably shows characteristics of the present embodiment, on the echo signal sₘ after the delay compensation to generate a beamformer representing an ultrasonic tomogram, and gives the beamformer to the display device 40. The signal processing unit 230 is, for example, a DSP (Digital Signal Processor), and is not shown in detail in FIG. 1, but a signal processing program is previously installed in the signal processing unit 230 to cause the signal processing unit 230 to execute beamforming processing based on the signal-to-noise ratio. The signal processing unit 230 executes signal-to-noise ratio beamforming or linear regression beamforming on the signal delayed by the receiving unit 220 according to the signal processing program. The beamforming processing to be executed by the signal processing unit 230 includes two kinds of the signal-to-noise ratio beamforming and the linear regression beamforming. Both of the signal-to-noise ratio beamforming and the linear regression beamforming are broadly based on the signal-to-noise ratio, but are given different names to distinguish between the two methods. The signal-to-noise ratio beamforming and the linear regression beamforming, which remarkably show the characteristics of the present embodiment, will be described below.

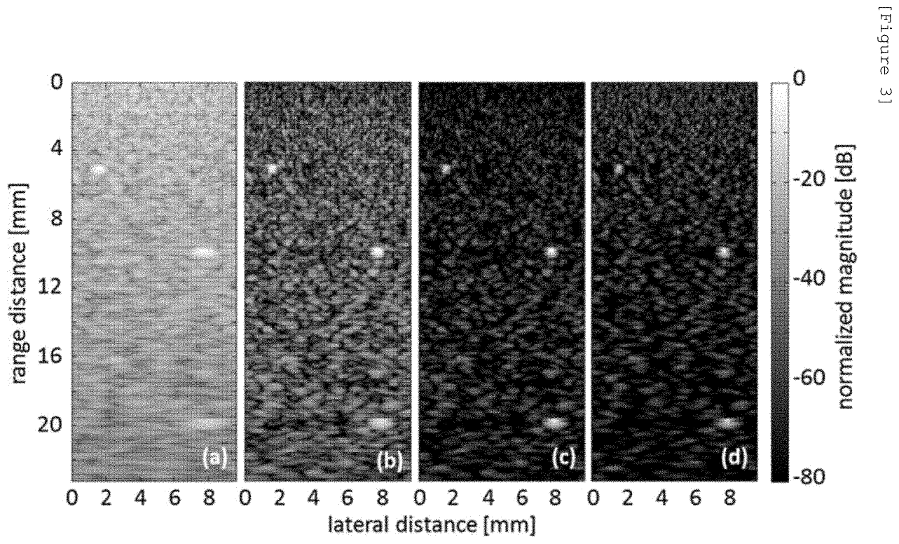

FIG. 2 is a flowchart showing a flow of the signal-to-noise ratio beamforming and the linear regression beamforming. As shown in FIG. 2, both methods include two steps of an estimation step SA100 and a generation step SA110 subsequent to the estimation step SA100. In other words, as shown in FIG. 1, the signal processing unit 230 operating according to the signal processing program functions as estimation means 230a for executing the estimation step SA100 and generation means 230b for executing the generation step SA110.

In the estimation step SA100 in the signal-to-noise ratio beamforming, the signal processing unit 230 estimates a signal-to-noise ratio in echo signals of M channels output from the receiving unit 220, and calculates a weight coefficient (a weight coefficient according to the signal-to-noise ratio) for emphasizing the echo from the reception focus. As described above, the echo y from the reception focus becomes a DC component of the echo signal sₘ after the delay compensation. In the estimation step SA100 in the signal-to-noise ratio beamforming, the signal processing unit 230 estimates a signal component and a noise component based on an average value and a variance of the echo signals sₘ after delay compensation, and calculates a weight coefficient W_{SNR}, which emphasizes the echo from the reception focus, according to Equation 3 to be described below. Then, in the generation step SA110 in the signal-to-noise ratio beamforming, the signal processing unit 230 calculates an output (that is, a beamformer representing an ultrasonic tomogram) Y_{SNR} of the signal-to-noise ratio beamforming according to Equation 4 to be described below, and gives the calculated output to the display device 40.

$$\begin{matrix}{w}_{SNR} = \frac{{\vert \frac{1}{M} {\sum }_{i = 0}^{M - 1} {s}_{i} \vert}^{2}}{\frac{1}{M} {\sum }_{i = 0}^{M - 1} {\vert {s}_{i} \vert}^{2} - {\vert \frac{1}{M} {\sum }_{i = 0}^{M - 1} {s}_{i} \vert}^{2}} & [ Equation 3 ] \\ {Y}_{SNR} = {W}_{SNR} \times {Y}_{DAS} & [ Equation 4 ]\end{matrix}$$

When the signal-to-noise ratio of the echo signal sₘ after the delay compensation is very high, the W_{SNR} becomes extremely large as the denominator in Equation 3 becomes very small, and the beamformer output becomes unstable. In order to avoid such a case, a stabilization parameter β (real number) may be introduced as in Equation 5.

$${w}_{SNR} = \frac{{\vert \frac{1}{M} {\sum }_{i = 0}^{M - 1} {s}_{i} \vert}^{2}}{\frac{1}{M} {\sum }_{i = 0}^{M - 1} {\vert {s}_{i} \vert}^{2} - β \cdot {\vert \frac{1}{M} {\sum }_{i = 0}^{M - 1} {s}_{i} \vert}^{2}} \qquad [ Equation 5 ]$$

As a value of β is closer to 0, the denominator in Equation 5 is avoided from becoming smaller, and the beamformer output becomes stable, but an improvement effect of spatial resolution is reduced. The value of the stabilization parameter β may be appropriately set to an appropriate value in consideration of the balance between the stability of the beamformer output and the improvement effect of spatial resolution. The contents of the signal-to-noise ratio beamforming are described above.

The linear regression beamforming will be described below.

In the estimation step SA100 in the linear regression beamforming, the signal processing unit 230 estimates noise in echo signals of M channels output from the receiving unit 220 and calculates a weight coefficient that emphasizes an echo from the reception focus, which are processing different from the processing in the signal-to-noise ratio beamforming. More specifically, the signal processing unit 230 first calculates a cumulative element signal uₘ according to Equation 6 to be described below (however, u₀=0). A symbol sᵢ on the right side in Equation 6 represents an echo signal after delay compensation from the i-th ultrasonic transducer.

$${u}_{m} = {\sum }_{i - 0}^{m - 1} {s}_{i} \qquad [ Equation 6 ]$$

As described above, the echo y from the reception focus becomes the DC component of the echo signal sₘ after the delay compensation. Therefore, the cumulative element signal uₘ is modeled as a linear function as indicated by Equation 7 to be described below. A symbol n in Equation 7 represents a bias caused by additional noise. In the following description, a signal modeled according to Equation 7 will be referred to as a modeling element signal. A mean squared error α between the measured cumulative element signal uₘ and the modeling element signal Uₘ is defined as in Equation 8 to be described below, and the signal processing unit 230 sets values of y and n (hereinafter, least-squares estimated values) such that the mean squared error α defined in Equation 8 is minimized (that is, estimates the signal-to-noise ratio). The least-squares estimated values of y and n are obtained when a partial differentiation of the mean squared error α to the values of y and n is set to zero, as indicated by Equation 9.

$$\begin{matrix}Um = y \times m + n & [ Equation 7 ] \\ α = {\sum }_{m = 0}^{M - 1} {\{ {u}_{m} - ( y \times m + n ) \}}^{2} & [ Equation 8 ] \\ ( Y , N ) = arg {min}_{y , n} α & [ Equation 9 ]\end{matrix}$$

Next, the signal processing unit 230 first substitutes least-squares estimated values Y and N calculated according to Equation 9 into the values of y and n in Equation 8 to calculate a minimum value αₘᵢₙ of the mean squared error α. Then, the signal processing unit 230 calculates a weight coefficient W_{LR}, which emphasizes the echo from the reception focus, according to Equation 10 to be described below, and ends the estimation step SA100 in the linear regression beamforming. In the generation step SA110 in the linear regression beamforming, an output of the linear regression beamformer (that is, a beamformer output representing the ultrasonic tomogram) Y_{LR} is calculated according to Equation 11 to be described below, and given to the display device 40.

$$\begin{matrix}{w}_{L , R} = \frac{{\vert Y \vert}^{2}}{{α}_{min}} & [ Equation 10 ] \\ {Y}_{LR} = {W}_{LR} \times Y & [ Equation 11 ]\end{matrix}$$

Similarly to the signal-to-noise ratio beamforming, when the signal-to-noise ratio of the echo signal sₘ after the delay compensation is very high, the W_{LR} becomes extremely large as the denominator in Equation 10 becomes very small, and the beamformer output becomes unstable. In order to avoid such a case, a stabilization parameter γ (real number) may be introduced as in Equation 12.

$${w}_{LR} = \frac{{\vert Y \vert}^{2}}{{α}_{min} + γ \cdot {\vert Y \vert}^{2}} \qquad [ Equation 12 ]$$

As a value of γ becomes larger, the beamformer output becomes stable, but an improvement effect of spatial resolution is reduced. Similarly to the stabilization parameter β, the value of the stabilization parameter γ may also be appropriately set to an appropriate value in consideration of the balance between the stability of the beamformer output and the improvement effect of spatial resolution. The contents of the linear regression beamforming are described above.

Since using the least squares method to estimate the signal-to-noise ratio, the estimation step SA100 in the linear regression beamforming described above has a high calculation load compared with the signal-to-noise ratio beamforming. Therefore, in order to improve calculation efficiency of the linear regression beamforming (that is, to reduce the calculation load), modifications may be applied as follows.

In the estimation step SA100 in the linear regression beamforming with improved calculation efficiency, the signal processing unit 230 calculates an integral value nₘ of noise included in a received signal sₘ by the m-th element using Equation 13.

$${n}_{m} = {\sum }_{i = 0}^{m} ( {s}_{i} - {Y}_{DAS} ) \qquad [ Equation 13 ]$$

A weight coefficient W_{LRe} in the linear regression beamforming with improved calculation efficiency is defined as Equation 14 to be described below, using the integral value nₘ of the noise component obtained by Equation 13. In the estimation step SA100 in the linear regression beamforming with improved calculation efficiency, the signal processing unit 230 calculates the weight coefficient W_{LRe} according to Equation 14. A symbol γ in Equation 14 represents a stabilization parameter similar to that in Equation 12.

$${w}_{LRe} = \frac{{\vert {Y}_{DAS} \vert}^{2}}{\frac{1}{M} {\sum }_{m = 0}^{M - 1} {\vert {n}_{m} \vert}^{2} + γ {\vert {Y}_{DAS} \vert}^{2}} \qquad [ Equation 14 ]$$

In the generation step SA110 in the linear regression beamforming with improved calculation efficiency, the signal processing unit 230 calculates a beamformer output Y_{LRe} representing an ultrasonic tomogram according to Equation 15 to be described below, and gives it the display device 40.

Y_{LRe}=W_{LRe}×Y_{DAS} [Equation 15]

Further, all of the signal-to-noise ratio beamforming, the linear regression beamforming, and the linear regression beamforming with improved calculation efficiency may reduce the amount of calculation by combining the aperture division processing disclosed in Non-Patent Literature 2.

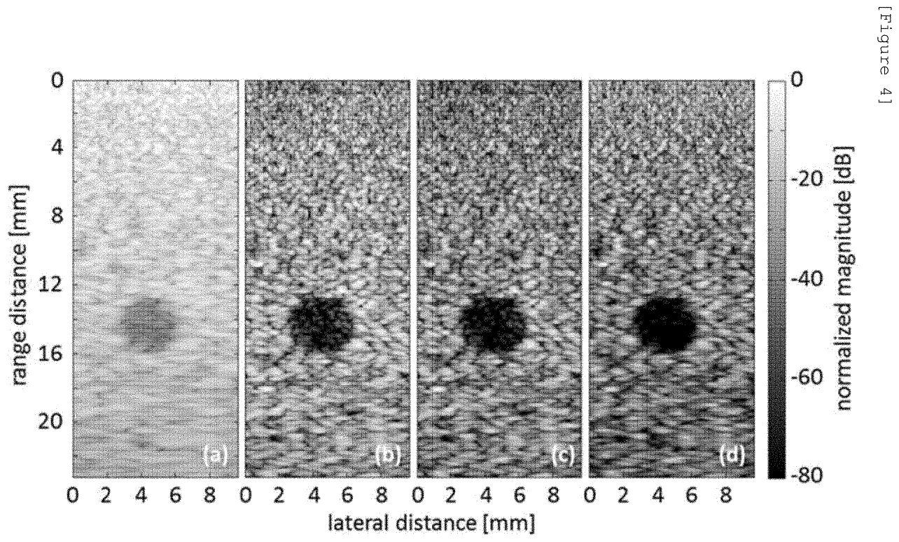

FIG. 3 shows an imaging result of a point target for evaluating spatial resolution of an ultrasonic tomogram. More specifically, FIG. 3(a) shows an image obtained by DAS beamforming, FIG. 3(b) shows an image obtained by a method based on coherence between received signals, and FIGS. 3(c) and 3(d) show images obtained by the signal-to-noise ratio beamforming and the linear regression beamforming of the present embodiment, respectively. In each of FIGS. 3(a) to 3(d), brightness (white intensity) of the image indicates intensity of ultrasonic scattered wave. As is clear from comparison of the images shown in FIGS. 3(a) to 3(d), the images (FIGS. 3(c) and 3(d)) obtained by the signal-to-noise ratio beamforming and the linear regression beamforming of the present embodiment has a white bright spot smaller than that of the images shown in FIGS. 3(a) and 3(b). From this fact, according to the signal-to-noise ratio beamforming and the linear regression beamforming of the present embodiment, higher spatial resolution can be obtained compared with the method based on the DAS beamforming and the coherence between the received signals.

FIG. 4 shows an imaging result of a phantom (virtual image) for evaluating a contrast of an ultrasonic tomogram. More specifically, FIG. 4(a) shows an image obtained by the conventional DAS beamforming, FIG. 4(b) shows an image obtained by the coherence between the received signals, and FIGS. 4(c) and 4(d) show images obtained by the signal-to-noise ratio beamforming and the linear regression beamforming of the present embodiment, respectively. In each of FIGS. 4(a) to 4(d), a dark portion in a central part is a medium (specifically, a cyst simulation part) in which ultrasonic scattered waves are not generated, and is preferably depicted in solid black. In each of the images shown in FIGS. 4(a) and 4(b), white bright spots are also generated in the cyst simulation part, and these white bright spots are virtual images. It can be seen in FIG. 4(c) that the virtual image is reduced. Further, in the image shown in FIG. 4(d), a virtual image is not depicted in the cyst simulation part. In other words, according to the signal-to-noise ratio beamforming and the linear regression beamforming of the present embodiment, it can be seen that the depiction of a virtual image in the cyst simulation part can be prevented and a contrast is improved compared with the method based on the conventional DAS beamforming and the coherence between the received signals.

In FIG. 4, the reason why the effect of preventing the virtual image is higher in the linear regression beamforming than in the signal-to-noise ratio beamforming is the effect of the processing of Equation 6, that is, the integration effect of the echo signal sₘ after the delay compensation. The integration corresponds to a low pass filter. It is possible to further improve the output of the linear regression beamformer by applying a filter other than the integration operation. Similarly, integration processing of Equation 13 may be appropriately changed to another filter processing.

As described above, according to the present invention, the spatial resolution and the contrast of the ultrasonic tomogram can be further improved compared with the method based on the conventional DAS beamforming and the coherence between the received signals.

### (B. Modifications)

Although the embodiment of the present invention has been described above, the following modifications may be added to the embodiment.

(1) In the embodiment, an example of the present invention applicable to the ultrasonic medical system is described, but the present invention can also be applied to generation of an ultrasonic tomogram for non-destructive inspection of an object other than medical use. This is because higher spatial resolution and contrast of the ultrasonic tomogram are more preferable even in technical fields other than the medical use.

(2) In the estimation step SA100 in the linear regression beamforming, the signal-to-noise ratio of the echo signals of the M channels is estimated by the least squares method, but the signal-to-noise ratio may be estimated by another method such as a method of using likelihood. In summary, there may be an ultrasonic tomogram generation method including: an estimation step of estimating noise in echo signals of M channels output from an ultrasonic probe, which receives echoes of ultrasonic waves emitted from M (being a natural number of 2 or more) ultrasonic transducers and outputs an echo signal, and calculating a weight coefficient for emphasizing an echo from a reception focus according to a signal-to-noise ratio in the echo signals of the M channels; and a generation step of generating a beamformer representing an ultrasonic tomogram from the echo signals of the M channels, using the weight coefficient calculated in the estimation step.

(3) In the above embodiment, the signal processing unit 230 of the ultrasonic tomogram generation apparatus 20 functions as the estimation means 230a and the generation means 230b, but the control unit 200 may function as the estimation means 230a and the generation means 230b. Specifically, the output signal of the receiving unit 220 may be given to the control unit 200, and the control unit 200 may execute the signal processing program of the embodiment described above.

(4) In the embodiment described above, the signal processing program for realizing the ultrasonic tomogram generation method, which remarkably show the characteristics of the present embodiment, is installed in advance in the ultrasonic tomogram generation apparatus 20. However, a program may be manufactured alone and distributed for sale, the program causing a computer such as a CPU to function as: estimation means for estimating noise in echo signals of M channels output from an ultrasonic probe, which includes M (being a natural number of 2 or more) ultrasonic transducers, receives echoes of ultrasonic waves emitted from the respective ultrasonic transducers, and outputs an echo signal, and calculating a weight coefficient for emphasizing an echo from a reception focus according to a signal-to-noise ratio in the echo signals of the M channels; and generation means for generating a beamformer representing an ultrasonic tomogram from the echo signals of the M channels, using the weight coefficient calculated by the estimation means. Specific examples of the distribution mode of the program include a mode in which the program is distributed by downloading via a telecommunication line such as the Internet and a mode in which the program is distributed in a state of being written in a computer-readable recording medium such as a CD-ROM (Compact Disk-Read Only Memory) or a flash ROM (Read Only Memory). When the computer is operated according to the program distributed in this way, the program can cause the computer to execute the ultrasonic tomogram generation method of the present invention.

(5) In the embodiment described above, the estimation means 230a and the generation means 230b for executing the respective steps of the ultrasonic tomogram generation method, which remarkably show the characteristics of the present embodiment, are implemented as software modules. However, an electronic circuit such as an ASIC may be used for each of estimation means for estimating noise in echo signals of M channels output from an ultrasonic probe, which includes M (being a natural number of 2 or more) ultrasonic transducers, receives echoes of ultrasonic waves emitted from the respective ultrasonic transducers, and outputs an echo signal, and calculating a weight coefficient for emphasizing an echo from a reception focus according to a signal-to-noise ratio in the echo signals of the M channels; and generation means for generating a beamformer representing an ultrasonic tomogram from the echo signals of the M channels, using the weight coefficient calculated by the estimation means, and these electronic circuits may be combined to form the ultrasonic tomogram generation of the present invention.

### REFERENCE SIGNS LIST

1 ultrasonic medical system
10 ultrasonic probe
20 ultrasonic tomogram generation apparatus
30 operating device
40 display device
200 control unit
210 transmission unit
220 receiving unit
230 signal processing unit
230a estimation means
230b generation means

1. An ultrasonic tomogram generation method comprising:
an estimation step of estimating noise in echo signals of M channels output from an ultrasonic probe, which receives echoes of ultrasonic waves emitted from M (being a natural number of 2 or more) ultrasonic transducers and outputs an echo signal, and calculating a weight coefficient for emphasizing an echo from a reception focus according to a signal-to-noise ratio in the echo signals of the M channels; and
a generation step of generating a beamformer representing an ultrasonic tomogram from the echo signals of the M channels, using the weight coefficient calculated in the estimation step.

2. The ultrasonic tomogram generation method according to claim 1, wherein
in the estimation step: a cumulative element signal uₘ is obtained by cumulation up to a m-th echo signal sₘ obtained by addition of a delay in delay-and-sum beamforming to the echo signals of the M channels; a modeling element signal is modeled as Uₘ=m×y+n using a direct component “y” included in the echo signal sₘ and a bias “n” caused by additional noise; values of the y and n are set such that a mean squared error α of the cumulative element signal and the modeling element signal is minimized; a minimum value of the mean squared error α is calculated using the set values of the y and n; and the weight coefficient is calculated from the minimum value and the set value of the y, and
in the generation step, the set value of the y is multiplied by the weight coefficient to generate the beamformer representing the ultrasonic tomogram.

3. The ultrasonic tomogram generation method according to claim 1, wherein
in the estimation step, the weight coefficient is calculated from a root mean square of an integral value nₘ of noise contained up to a m-th echo signal sₘ obtained by addition of a delay in delay-and-sum beamforming to the echo signals of the M channels and a root mean square of an average Y_{DAS} of the echo signal sₘ after delay compensation, and
in the generation step, the average Y_{DAS} is multiplied by the weight coefficient to generate the beamformer representing the ultrasonic tomogram.

4. An ultrasonic tomogram generation apparatus comprising:
estimation means for estimating a signal-to-noise ratio in echo signals of M channels output from an ultrasonic probe, which includes M (being a natural number of 2 or more) ultrasonic transducers, receives echoes of ultrasonic waves emitted from the respective ultrasonic transducers, and outputs an echo signal, and calculating a weight coefficient for emphasizing an echo from a reception focus according to a signal-to-noise ratio in the echo signals of the M channels; and
generation means for generating a beamformer representing an ultrasonic tomogram from the echo signals of the M channels, using the weight coefficient calculated by the estimation means.

5. A program causing a computer to function as:
estimation means for estimating a signal-to-noise ratio in echo signals of M channels output from an ultrasonic probe, which includes M (being a natural number of 2 or more) ultrasonic transducers, receives echoes of ultrasonic waves emitted from the respective ultrasonic transducers, and outputs an echo signal, and calculating a weight coefficient for emphasizing an echo from a reception focus according to a signal-to-noise ratio in the echo signals of the M channels; and
generation means for generating a beamformer representing an ultrasonic tomogram from the echo signals of the M channels, using the weight coefficient calculated by the estimation means.
